(19) United States
(12) Patent Application Publication
(10) Pub. No.: US 2006/0177926 A1
(43) Pub. Date: Aug. 10, 2006

(54) Common marmoset embryonic stem cell lines

(76) Inventors: Erika Sasaki, Tokyo (JP); Yoshikuni Tanioka, Kanagawa (JP); Kenzaburo Tani, Tokyo (JP); Hideyuki Okano, Tokyo (JP)

Correspondence Address: HELLER EHRMAN LLP, 275 MIDDLEFIELD ROAD, MENLO PARK, CA, 94025-3506, US

(21) Appl. No.: 11/218,699

(22) Filed: Sep. 2, 2005

Related U.S. Application Data
(60) Provisional application No. 60/607,864, filed on Sep. 7, 2004.

(51) Int. Cl. C12N 5/06

(52) U.S. Cl. 435/363

(57) ABSTRACT

The present invention relates to novel common marmoset (Callithrix jacchus) embryonic stem cell cultures and cell lines, and their use. The common marmoset embryonic cells of the present invention (i) are capable of prolonged undifferentiated proliferation in vitro, (ii) maintain, during prolonged culture, a karyotype in which all the chromosomal characteristics of common marmoset are present without noticeable alteration; (iii) are capable of differentiation into all three embryonic germ layers (ectoderm, endoderm and mesoderm) even after prolonged culture; and (iv) are capable of teratoma formation in vivo. The stem cells preferably are totipotent.

### BACKGROUND OF THE INVENTION

1. Field of the Invention

The present invention relates to novel common marmoset (Callithrix jacchus) embryonic stem cell cultures and cell lines, and to their use.

2. Related Art

Embryonic stem cells (ES cells) are derived from totipotent cells from early mammalian embryo and are capable of unlimited undifferentiated proliferation in vitro (Evans and Kaufmnan, Nature 292:154 (1981); Martin, G., Proc. Natl. Acad. Sci. USA 78:7634 (1981). ES cells can differentiate into any cell type in vivo (Evans et al., Nature 292:154-156 (1981); Bradley, et al., Nature 309:255-256 (1984)), and into a variety of cells in vitro (Doetschman, et al., J. Embryol. Exp. Morph., 87:27-45 (1985); Wobus, et al., Biomed. Biochim. Acta, 47:965-973 (1988); Robbins, et al., J. Biol. Chem., 265:11905-11909 (1990)). Non-human primate and human embryonic stem cell lines have been established (see, e.g. Thomson and Marshall, Curr Top Dev Biol 38:133-65 (1998); Thomson et al., Proc Natl Acad Sci USA 92:7844-7848 (1995); and Thomson et al. Science 282:1145-1147 (1998)). Primate embryonic stem cells are described, for example, in U.S. Pat. No. 5,843,780 issued on Dec. 1, 1998. Monkey-origin embryonic stem cells are disclosed in U.S. Patent Application Publication No. 2003/0157710 A1, published on Aug. 21, 2003.

According to Thomson et al., PNAS, supra, the essential characteristics of primate ES cells should include (i) derivation from the preimplantation or periimplantation embryo, (ii) prolonged undifferentiated proliferation, and (iii) stable developmental potential to form derivatives of all three embryonic germ layers (ectoderm, endoderm and mesoderm) even after prolonged culture. The present invention provides marmoset-derived embryonic cells and cell lines, which possess such characteristics.

### SUMMARY OF THE INVENTION

The invention concerns marmoset (Callithrix jacchus) derived embryonic cell preparations and cell lines.

In one aspect, the invention concerns a purified preparation of embryonic stem cells derived from common marmoset (Callithrix jacchus), which (i) is capable of prolonged undifferentiated proliferation in vitro, (ii) maintains, during prolonged culture, a karyotype in which all the chromosomal characteristics of common marmoset are present without noticeable alteration; (iii) is capable of differentiation into all three embryonic germ layers (ectoderm, endoderm and mesoderm) even after prolonged culture; and (iv) is capable of teratoma formation in vivo.

In a preferred embodiment, the common marmoset-derived embryonic stem cells are totipotent.

In one embodiment, in the purified preparation the embryonic stem cells are positive for the SSEA-3, SSEA-4, TRA1-60, and TRA1-81 markers, and negative for the SSEA-1 marker.

In another embodiment, in the purified preparation the embryonic stem cells are positive for the Neuronal Class 3 Beta Tubulin and Oligodendrocyte Marker O4 markers.

In yet another embodiment, in the purified preparation the embryonic stem cells exhibit high telomerase activity.

In a further embodiment, in the purified preparation the embryonic stem cells are capable of undifferentiated proliferation in vitro for at least about 3 months, or at least about 6 months, or at least about 8 months, or at least about 10 months, or at least about one year, or at least about 18 months, or at least about two years.

In a still further embodiment, in the purified preparation the embryonic stem cells are capable of differentiation into all three embryonic germ layers (ectoderm, endoderm and mesoderm) after continuous culture for at least about 3 months, or at least about 6 months, or at least about 8 months, or at least about 10 months, or at least about one year, or at least about 18 months, or at least about two years.

In another aspect, the invention concerns cell line developed from embryonic stem cells of common marmoset (Callithrix jacchus), which (i) is capable of prolonged undifferentiated proliferation in vitro, (ii) maintains, during prolonged culture, a karyotype in which all the chromosomal characteristics of common marmoset are present without noticeable alteration; (iii) is capable of differentiation into all three embryonic germ layers (ectoderm, endoderm and mesoderm) even after prolonged culture; and (iv) is capable of teratoma formation in vivo.

Preferably, the cell line is totipotent.

In one embodiment, the cell line is positive for the SSEA-3, SSEA-4, TRA1-60, and TRA1-81 markers, and negative for the SSEA-1 marker.

In another embodiment, the cell line is positive for the Neuronal Class 3 Beta Tubulin and Oligodendrocyte Marker O4 markers.

In a different aspect, the invention concerns a method of making a genetically modified common marmoset (Callithrix jacchus), comprising

(a) introducing a mutation into a common marmoset stem cell having the characteristics described above, and

(b) transplanting the stem cell into the common marmoset.

The method may further comprise the step of analyzing the differentiation and proliferation of the stem cells in the common marmoset.

In a particular embodiment, the stem cells may carry one or more foreign genes and/or may have one more endogenous genes knocked out and/or may carry one or more spontaneous mutations.

In another aspect, the invention concerns a common marmoset (Callithrix jacchus) carrying a genetic modification resulting from the introduction into such common marmoset genetically modified common marmoset embryonic stem cells which (i) are capable of prolonged undifferentiated proliferation in vitro, (ii) maintain, during prolonged culture, a karyotype in which all the chromosomal characteristics of common marmoset are present without noticeable alteration; (iii) are capable of differentiation into all three embryonic germ layers (ectoderm, endoderm and mesoderm) even after prolonged culture; and (iv) are capable of teratoma formation in vivo.

In a further aspect, the invention concerns a stem cell assay method comprising transplanting human stem cells to a common marmoset (Callithrix jacchus) animal model of a disease or condition susceptible to cell based therapy, and monitoring the response of said common marmoset to the transplantation of said human stem cells.

In another aspect, the invention concerns an assay method comprising transplanting human stem cells to a common marmoset (Callithrix jacchus), and analyzing the differentiation and proliferation of said human stem cells in said common marmoset.

In yet another aspect, the invention concerns an immunodeficient rodent having embryonic stem cells of common marmoset (Callithrix jacchus) transplanted therein, wherein the cells (i) are capable of prolonged undifferentiated proliferation in vitro, (ii) maintain, during prolonged culture, a karyotype in which all the chromosomal characteristics of common marmoset are present without noticeable alteration; (iii) are capable of differentiation into all three embryonic germ layers (ectoderm, endoderm and mesoderm) even after prolonged culture, and (iv) are capable of teratoma formation in vivo.

Preferably, the transplanted stem cells are totipotent.

The rodent can, for example, be a rat, a mouse, or a guinea pig, such as an athymic nude mouse, C.B-17/severe combined immunodeficiency (scid) mouse or NOD/SCID mouse.

The rodent, such as a mouse, can be a model of a human disease or condition potentially susceptible to cell based therapy. Such diseases and conditions include, without limitation, Parkinson's disease, diabetes, traumatic spinal cord injury, Purkinje cell degeneration, Duchenne's muscular dystrophy, heart disease, vision loss and hearing loss.

The rodent, such as rat or mouse, can have one or more mutations, in addition those required to provie immunodeficiency, which result in modeling a target human disease or condition. The mutations may include spontaneous mutations, the introduction of one or more transgenes or knocking out one or more endogenous genes, and any combinations thereof.

The stem cells can be in an undifferentiated form, in the form of embryoid bodies, and/or in a differentiated form.

### BRIEF DESCRIPTION OF THE DRAWINGS

FIG. 1. Expression of alkaline phosphatase and cell surface markers on CMES cells. (A), (B) Unstained CMES cells; (C) cells stained for alkaline phosphatase; (D) SSEA-1; (E) SSEA-3; (F) SSEA-4; (G) TRA1-60; and (H) TRA1-81.

FIG. 2. Karyotype analysis of CMES cells. The results for CIEA-CMES #10 are shown. All three cell lines show the Normal 46, XX karyotype after more than six months of culture.

FIG. 3. (A) Telomerase activities of CMES cell lines Nos. 20, 30, and 40. All three CMES lines show high teloperase activity. On the other hand, MEF does not show Telomerase activity. (B) SSCP analysis of CMES cells for the MHC-DRB1 gene. The results of SSCP indicate that all three lines were derived independently.

FIG. 4. (A) RT-PCR analysis of CMES cells and embryoid bodies. Undifferentiated CMES cells expressed the Nanog, Oct3/4, Sox2, FoxD3, Bex1/Rex3, Heb and mCG, GP130 and low level of Nestin genes. Two-week cultures of embryoid bodies display expression of Nestin and CD34. Three-week cultures of embryoid bodies show expression of Nestin, CD34, and α-fetoprotein. Oct3/4 and Nanog gene expression are shut down in the embryoid bodies. In undifferentiated CMES cells expressed. (B) RT-PCR analysis of fresh ICMs. ICMs expressed the Nanog, Oct3/4, Sox2 genes. Expression of FoxD3 and Nestin were not detected.

FIG. 5. Spontaneous differentiation potency of CMES cells. A suspension culture of CMES shows the formation of embryoid bodies (EBs): (A) simple EB; (B) cystic EB.

FIG. 6. Differentiated CMES cells in teratomas, and the expression of tissue-specific markers. (A) A bronchus-like structure that consists of columnar epithelium surrounded by cartilage-like tissue is shown. (B) Keratinizing squamous epidermis; (c) striated muscle; (D) adipose-like tissue (high magnification); (E) cartilage-like and columnar epithelium (high magnification); (F) epidermis; (G) reactivity for cytokeratin WSS; (H) muscle expressing desmin; (I) capillary blood vessels; (J) CD31-positive vascular endothelial cells; (K) columnar epithelium; (L) Alcian blue-PAS positive columnar epithelia; (M) GFAP-expressing neural cells.

FIG. 7. Hematopoietic CMES cell differentiation in vivo. (A) CFU-M monocyte/macrophage-like colonies predominate under these conditions. (B) May-Giemsa staining of colony forming cells, confirming that the major population consists of macrophages.

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

A “blastocyst” is a preimplantation embryo of about 150 cells. The blastocyst consists of a sphere made up of an outer layer of cells (the trophectoderm), a fluid-filled cavity (the blastocoel), and a cluster of cells on the interior (the inner cell mass).

“Multipotent” stem cells are stem cells that can reproduce only limited cells and tissues in the body.

“Pluripotent” stem cells are stem cells that can reproduce every cell and tissue in the body, except the placenta. Whether embryonic stem cells are pluripotent is typically tested by allowing the cells to differentiate spontaneously in cell culture, manipulating the cells so that they differentiate to form specific cell types, or injecting the cells into an immunosuppresed mouse to test for the formation of a benign tumor called a teratoma. Teratomas typically contain a mixture of many differentiated or partly differentiated cell type, which is an indication that the embryonic stem cells are capable of differentiating into multiple cell types.

“Totipotent” stem cells are stem cells that can reproduce every cell and tissue in the body, including the placenta. Such stem cells can give rise to the entire organism, including the extra-embryonic membranes. Thus, e.g. the fertilized egg or zygote is totipotent.

The term “undifferentiated” is used herein to refer to cells which have not changed to become a specialized cell type to any significant degree.

The term “cell based therapy” is used herein to refer to any therapy which involves the induction of stem cells to differentiate into a specific cell type required to achieve the desired therapeutic effect, such as, to repair damaged or repleted adult cell populations or tissues.

### DETAILED DESCRIPTION

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Animal Cell Culture” (R. I. Freshney, ed., 1987); “Gene Transfer Vectors for Mammalian Cells” (J. M. Miller & M. P. Calos, eds., 1987); “Current Protocols in Molecular Biology” (F. M. Ausubel et al., eds., 1987); and “Embryonic Stem Cells: Methods and Protocols” (Kursad Turksen, ed., Humana Press, Totowa N.J., 2001.

Since the successful establishment of human embryonic stem (ES) cell lines in 1998, (Thomson et al, Science 282:1145-1147 (1998)) transplantation of differentiated embryonic stem cells to specific organs has been expected to have great therapeutic potentials. It has been, however, very difficult to confirm the safety and efficacy of human embryonic cell based therapy (stem cell therapy) in vivo, due to ethical regulations restricting human experimentation. Therefore, it is essential to establish animal models using non-human primates for preclinical studies.

The common marmoset (Callithrix jacchus) is a New World primate species. The animals are small (weighing about 350-400 g), have a short gestation period (about 114 days) and reach sexual maturity at 12-18 months. Unlike macaques, marmosets routinely deliver twins or triplets for each pregnancy. In addition, it is possible to synchronize the marmoset ovarian cycle with prostaglandin analogs, collect age-matched embryos from multiple females, and transfer embryos to synchronized recipients with success rates in the range of 70-80% (Lopata et al., Fertil Steril 50:503-509 (1988); Summers et al., J Reprod Fertil 79:241-250 (1987); and Marshall et al., J Med Primatol 26:241-247 (1997)). Since these reproductive characteristics allow routine efficient transfer of multiple embryos, marmosets constitute an excellent primate species for the generation of transgenic and knockout animal models of human diseases.

The present invention is based on the establishment of novel (totipotent) common marmoset embryonic cell lines (CMES cell lines), which facilitate the construction, by gene targeting, of non-human primate animal models for various human diseases.

Following the expression of molecular markers characteristic of undifferentiated human embryonic stem cells, we have demonstrated that the hematopoietic and immune systems of common marmosets are very similar to those of human. Thus, SSEA-1, a carbohydrate antigen, is a fucosylated derivative of type 2 polylactosamine and is known to appear during late cleavage stages of mouse embryos. It is strongly expressed by undifferentiated, murine ES cells. Upon differentiation, murine ES cells are characterized by the loss of SSEA-1 expression and may be accompanied, in some instances, by the appearance of SSEA-3 and SSEA-4. In contrast, human ES and EC cells typically express SSEA-3 and SSEA-4 but not SSEA-1, while their differentiation is characterized by down-regulation of SSEA-3 and SSEA-4 and an up-regulation of SSEA-1. Undifferentiated, human ES cells also express the keratan sulphate-associated antigens, TRA-1-60 and TRA-1-81. The immunohistochemistry studies described in the Example below demonstrate that, similarly to human embryonic stem cells, undifferentiated common marmoset embryonic stem cells (CMES) express SSEA-3, SSEA-4, TRA-1-60 and TRA-1-81, and do not express SSEA-1. Accordingly common marmosets are excellent laboratory animal models for the pre-clinical study of stem cell therapy. This is particularly true since it is comparatively easy to handle common marmosets as laboratory animals due to their size.

Thus, common marmoset (CM) ES cell lines are powerful tools for producing genetically modified common marmosets for use as primate animal models of various human diseases, as well as understanding the regulating mechanisms of ES cell differentiation in vitro and in vivo.

The CMES cell lines are additionally useful for producing specific cell types, such as endothelial cells, neuron cells, blood cells, muscle cells, and the like, which can be used for tissue transplantation.

The CMES cell lines can be further used to produce tumors in various mouse models, such as, for example, SCID, NOD/SCID, and NOD/SCID/γ_{c}^{null} mice (see, e.g. U.S. Patent Publication No. 20030182671; Dewan et al., J. Virol. 77(9):5286-94 (2003); and Miyakawa et al., Biochem. Biophys. Res. Commun. 313(2):258-62 (2004), the entire disclosures of which are hereby expressly incorporated by reference). The tumors can then be disassociated, and the desired cell types selected by using of lineage-specific markers, for example, by using fluorescent activated cell sorting (FACS), or by direct microdissection of the desired tissues. The differentiated cells can be reintroduced into adult animals to treat various diseases, such as neurological, endocrine, or hematopoietic disorders.

Some methods of the present invention involve the introduction of foreign genes into stem cells. Gene delivery most commonly is performed using retroviral vectors by techniques well known in the art. Retroviruses are enveloped viruses containing a single stranded RNA molecule as their genome. Following infection, the viral genome is reverse transcribed into double stranded DNA, which integrates into the host genome where it is expressed. The viral genome contains at least three genes: gag (coding for core proteins), pol (coding for reverse transcriptase) and env (coding for the viral envelope protein). At each end of the genome are long terminal repeats (LTRs) which include promoter/enhancer regions and sequences involved with viral integration. In addition there are sequences required for packaging the viral DNA and RNA splice sites in the env gene. Retroviral vectors used in mouse models are most frequently based upon the Moloney murine leukemia virus (Mo-MLV). In addition, lentiviruses can, for example, be used for gene transfer into experimental animals, such as NOG mice.

Gene delivery can also be performed by adenoviral vectors. Adenoviroses are non-enveloped, icosahedral viruses with linear double-stranded DNA genomes. Adenoviruses infect non-dividing cells by interacting with cell surface receptors, and enter cells by endocytosis. Since the genome of adenoviruses cannot integrate with the host cell genome, the expression from adenoviral vectors is transient.

The present invention enables the study and development of new cell based therapies for the treatment of a variety of human diseases and conditions. Such treatments are based on the use of stem cells, progenitor cells or differentiated cells for the treatment of heart, lung, blood, neural, sickle cell diseases, hearing impairments, sleep disorders, and the like, and hold enormous potential in regenerative medicine, including targeting Parkinson's disease, Alzheimer's disease, ageing, and Type I diabetes.

Further details of the invention are illustrated by the following non-limiting example.

### EXAMPLE

Materials and Methods

Animals

To obtain marmoset embryos, 15 pairs of common marmosets (over 2 years of age) were selected from an experimental animal breeding colony sustained since 1975. Each pair of animals was caged separately. The cage size was 39×60×70 cm. The study was approved by the animal ethics committees of CIEA, and was performed in accordance with CIEA guidelines.

Embryo Collection

Fifteen female animals were divided into three groups. The ovulation cycles of each group of animals were synchronized with a prostaglandin (PG) F2α analog, cloprostenol (0.75 mg/head Estrumate®, Takeda Schering-Plough K. K., Osaka, Japan), which was administered more than 10 days after the luteal phase, as reported previously (Summers, P. M., J Reprod Fertil 73:133-8 (1985)). Plasma samples (0.1 ml) were collected from the femoral vein at 2, 9, 10 and 13 days after cloprostenol injection, and the day of ovulation was determined by measuring plasma progesterone concentration using an EIA assay, as described below. The day of ovulation (Day 0) was defined as the day before the serum progesterone level reached 10 ng/ml (Harlow, C. R., J Zool Lond 201:273-282 (1983)). Embryos were surgically collected 7-10 days after ovulation (Summers, P.M., J Reprod Fertil 79:241-50 (1987)), following anesthesia by intramuscular injection of 0.5 mg/head of medetomidine hydrochloride (Domitor®, Meiji, Tokyo, Japan) or 0.25-0.5 mg/head of flunitrazepam (Silece®, Eisai, Tokyo, Japan), and 70 mg/head of ketamine hydrochloride (veterinary Ketalar 50, Sankyo Lifetech Co., Tokyo, Japan). The cervix and both oviducts were exteriorized by midline laparotomy and clamped, and the uterine lumen was flushed from the proximal end to the cervix with 2.5 ml of Dulbecco's-modified Eagle's medium (DMEM; Invitrogen, Tokyo, Japan) that contained 10% fetal bovine serum (FBS; JRH, Tokyo, Japan). The flushed medium was collected using a 23-gauge needle that was placed in the uterine lumen through the uterine fundus. Cloprostenol was also administered using the DPC Progesterone Kit (Diagnostic Products Corp., Los Angeles, CA), according to the recommendations of the manufacturer.

Isolation and Culture of Embryonic Stem Cell Lines

Inner cell masses (ICMs) were isolated by immunosurgery, as described previously (Solter and Knowles, Proc Natl Acad Sci USA 72:5099-5102 (1975)). Briefly, the zona pellucida of the marmoset blastocysts were washed three times with DMEM. To remove the trophectoderm, the blastocysts were incubated for 45 min at 37° C. in 5% CO₂ with a 10-fold dilution of anti-marmoset fibroblast rabbit serum in DMEM. Following three washes with DMEM, the blastocysts were incubated with a 5-fold dilution of Guinea pig complement (Invitrogen, Tokyo, Japan) in DMEM for 30 min at 37° C. in 5% CO₂. After immunosurgery, the trophectoderm was removed by pipetting, and the ICMs were isolated. The ICMs were plated on 3500 rad γ-irradiated mouse embryonic feeder layer (MEF). After 10-14 days, the ICMs were dissociated in trypsin-EDTA and re-plated on a fresh MEF layer. The ICMs and their expanded cells were cultured using CMES medium that consisted of 80% Knockout DMEM supplemented with 20% Knockout Serum Replacement (KSR; Invitrogen), 1 mM L-glutamine, 0.1 mM MEM non-essential amino acids, 0.1 mM β-mercaptoethanol (2-ME; Stigrna, Tokyo, Japan), 100 IU/ml penicillin, 100 μg/ml streptomycin sulfate, 250 ng/ml amphotericin B, and 10 ng/ml leukemia inhibitory factor. For cell splitting, undifferentiated CMES cell colonies were detached from the feeder cells using 0.25% trypsin that was supplemented with 1 mM CaCl₂ and 20% KSR. The removed colonies were mechanically dissociated into 10-50 cells and re-plated on new irradiated MEF.

Immunohistochemical Staining

To examine the expression of cell surface markers, alkaline phosphatase was detected using the Alkaline Phosphatase Staining Kit (Sigma, Tokyo, Japan) following manufacturer's instructions. For immunostaining, ES cells were fixed with 4% paraformaldehyde in phosphate-buffered saline (PBS) for 10 minutes at room temperature, and then incubated with 0.3% H₂O₂ for 10 minutes at room temperature. The primary antibodies against stage-specific embryonic antigens SSEA-1, SSEA-3, SSEA-4 (Developmental Studies Hybridoma Bank), TRA-1-60, and TRA-1-81 (Chemicon, Temecula, CA) were diluted with Antibody Diluent (DAKO ChemMate, Dako, Glostrup, Denmark) and then incubated for 1hr at room temperature. The following primary antibodies (dilution) were used: anti-SSEA-1(1:50); anti-SSEA-3(1:10); anti-SSEA-4(1:50); anti-TRA1-60 and anti-TRA1-81 (10 μg/ml). After three washes with PBS, the biotinylated secondary antibody Simple Stain PO Multi system (Nichirei Corporation, Tokyo, Japan) was incubated with the cells for 30 min at room temperature. The samples were washed three times with PBS, and the localization of the bound monoclonal antibodies was detected using the DAB horseradish peroxidase complex.

For immunohistochemical analysis of tumors that formed after transplantation into immunodeficient mice, the collected tumors were fixed in neural buffered formalin, and embedded in paraffin. The paraffin blocks were sectioned and subjected to immunohistochemical staining. Primary antibodies against keratin (WSS), desmin, CD31, and GFAP (all purchased from DakoCytomation, Tokyo, Japan) were incubated with the paraffin sections at dilutions of 1:200, 1:200, 1:10, and 1:50, respectively. The localization of the bound monoclonal antibodies was detected using the Envision System (DakoCytomation).

For immunofluorescence staining of in vitro differentiated neural cells, the slides and sections were pre-incubated with 10% normal got serum plus 0.3% Triton X-100 in PBS, followed y overnight incubation in 10% normal goat serum plus 0.3% Triton -100 in PBS that contained rabbit polyclonal anti-TH antibody (Chemicon AB152). Alexa™ 488 goat anti-rabbit IgG antibody and AlexaTm 568 goat anti-mouse IgG antibody (Jackson ImmunoResearch, West Grove, Pa.) were added as secondary antibodies for 2 hours. Finally, the specimens were soaked in 2 μg/ml Hoechst 33528 in distilled water. All of the micrographs were analyzed on the Zeiss-Axiocam imaging system.

Karyotypic Analysis

ES cells were prepared by passaging a confluent culture from a 25-cm² bottle. After a 3-hour incubation period with fresh medium, a colcemid (Invitrogen, Tokyo, Japan) was added to a final concentration of 0.02 μg/ml for 20 minutes. The cells were then washed in PBS, dissociated using trypsin, and spun down. The pellet was resuspended carefully in 0.56% KCl at room temperature. After centrifugation, the hypotonic solution was removed, and the pellet was fixed with methanol/acetic acid, 3:1(vol/vol) via gently pipetting. After centrifuging at 100 rpm, 5 min fixation was performed twice before spreading the cells on slides. The slides were aid-dried overnight, stained in freshly made 5% Giemsa for 10 minutes, and rinsed with distilled water. In the Giemsa (G)-banding analysis, the numbers of chromosomes as well as karyotypic analysis were performed using 30 and 5 metaphase spreads, respectively.

Telomerase Activity

Telomerase activity was determined using the TRAPEZE Telomerase Detection Kit (Chemicon, Tokyo, Japan). All procedures followed manufacturer's instructions. Briefly, cell extracts were obtained from approximately 1×10⁶ cells, and the protein concentrations were normalized using the Coomassie blue-stained protein assay reagent BSA standards (Pierce Inc., Rockford, Ill.). Heat-inactivated controls were obtained by incubating the samples at 85° C. for 10 min. Aliquots (1.5 μg) of the cell extracts were used for PCR, which was performed according to the manufacturer's instructions. The PCR products were electrophoresed on a 12.5% non-denaturing polyacrylamide gel, and Telomerase activity was detected by SYBR green staining (Invitrogen).

PCR-Single Strand Conformation Polymorphism (PCR-SSVP)

SSCP analysis of the major histocompatibility complex-DRB genes was performed as described previously (Wu et al., J Mol Evol 51:214-222 (2000)). The following PCR primers were used: MA-DR-2r,

| | |
|---|---|
| 5′-CTCTCCGCGGCACTAGGAAC-3′ | (SEQ ID NO: 1) |
| 5′-GCACGTTTCTTGGAGTATAGC-3′ | (SEQ ID NO: 2) |

RT-PCR

Poly(A)³⁰ RNA was isolated using the QuickPrep Micro mRNA Purification Kit (Amersham Biotech, Buckinghamshire, UK) according to the manufacturer's instructions. First-strand cDNA was synthesized from 1 μg of poly(A)³⁰ RNA from the undifferentiated ES cells, or the EBs, using the ImProm-II cDNA Synthesis Kit (Promega, Tokyo, Japan). As negative controls, 1 μg of the poly(A)³⁰ RNA was allowed to react with the cDNA synthesis reaction mixture in the absence of ImProm-II reverse transcriptase. Following cDNA synthesis, 1/20 of the cDNA synthesis reaction mixture was used as the template for the PCR. For RT-PCR analysis of fresh ICMs, ICMs were obtained from seven blastocysts and used for poly(A)³⁰ RNA isolation. Half of the isolated poly(A)³⁰ RNA was then used for first-strand cDNA synthesis, and the other half was used as a negative control, as described above.

Individual primers were designed for the target genes. The following (forward and reverse) primer pairs were used.

| | |
|---|---|
| Nanog: | |
| 5′-AAACAGAAGACCAGAACTGTG-3′ | (SEQ ID NO: 3) |
| and | |
| 5′-AGTTGTTTTTCTGCCACCTCT-3′; | (SEQ ID NO: 4) |
| Oct3/4: | |
| 5′-CCTGGGGGTTCTATTTGGGA-3′ | (SEQ ID NO: 5) |
| and | |
| 5′-TTTGAATGCATGGGAGAGCC-3′; | (SEQ ID NO: 6) |
| FoxD3: | |
| 5′-CGACGACGGGCTGGAGGAGAA-3′ | (SEQ ID NO: 7) |
| and | |
| 5′-ATGAGCGCGATGTACGAGTA-3′; | (SEQ ID NO: 8) |
| Sox-2: | |
| 5′-AGAACCCCAAGATGCACAAC-3′ | (SEQ ID NO: 9) |
| and | |
| 5′-GGGCAGCGTGTACTTATCCT-3′; | (SEQ ID NO: 10) |
| CD34: | |
| 5′-AGCCTGTCACCTGGAAATGC-3′ | (SEQ ID NO: 11) |
| and | |
| 5′-CGTGTTGTCTTGCTGAATGGC-3′; | (SEQ ID NO: 12) |
| Nestin: | |
| 5′-GCCCTGACCACTCCAGTTTA-3′ | (SEQ ID NO: 13) |
| and | |
| 5′-GGAGTCCTGGATTTCCTTCC-3′; | (SEQ ID NO: 14) |
| α-fetoprotein: | |
| 5′-GCTGGATTGTCTGCAGGATGGGGAA-3′ | (SEQ ID NO: 15) |
| and | |
| 5′-TCCCCTGAAGAAAATTGGTTAAAAT-3′; | (SEQ ID NO: 16) |

marmoset chorionic gonadotropin (mCG) β:

| | |
|---|---|
| 5′-CCCTGTGTGTGTCGCCTTT-3′; | (SEQ ID NO: 17) |
| 5′-CTAATGGAGGGTCTGCTGGC-3′; | (SEQ ID NO: 18) |
| Bex1/Rex3: | |
| 5′-ACAGGCAAGGATGAGAGAAG-3′ | (SEQ ID NO: 19) |
| and | |
| 5′-CCCACGTAAACAAGTGACAG-3′; | (SEQ ID NO: 20) |
| HEB: | |
| 5′-ACTGAAACAAAGAAAGGATGAAAACC-3′ | (SEQ ID NO: 21) |
| and | |
| 5′-CCCTTTCTATCTTCTGTTCAGGGTTC-3′; | (SEQ ID NO: 22) |
| gp130: | |
| 5′-AAACAGAACAGCATCCAGTC-3′ | (SEQ ID NO: 23) |
| and | |
| 5′-AGTTGAGGCATCTTTGGTCC-3; | (SEQ ID NO: 24) |

Leukemia Inhibitory Factor Receptor (LIFR):

| | |
|---|---|
| 5′-TTTCTTGGCATTTACCAGG-3′ | (SEQ ID NO: 25) |
| and | |
| 5′-GCTATTTTGGAAGGTGGTG-3′; | (SEQ ID NO: 26) |
| β-actin: | |
| 5′-TCCTGACCCTSAAGTACCCC-3′ | (SEQ ID NO: 27) |
| and | |
| 5′-GTGGTGGTGAAGCTGTAGCC-3′. | (SEQ ID NO: 28) |

Except for CD34, α-fetoprotein. and mCF, the expected sizes of the PCR products were estimated from human sequences. The expected PCR products were ˜190 bp (nanog); ˜530 bp (oct3/4); ˜200 bp (Sox-2); ˜356 bp (FoxD3); ˜200 bp (nestin) 627 bp (CD34); 200 bp (α-fetoprotein), ˜559 bp (gp130); ˜269 bp (Bex1/Rex3); ˜165 bp (HEB); 286 bp (mCG); ˜514 bp (LIFR); and 418 bp (β-actin). The PCR reaction mixture (25 μl) contained 1×PCR buffer (10 mM Tris-HCl (pH 9.0), 1.5 MgCl₂, 50 mM KCl), 0.2 mM dNTP, 0.5 μg of each primer, and 2.5 U Taq polymerase. The amplification was performed for 35 cycles of denaturation at 95° C. for 1 minute, annealing at 60° C. for 30 seconds, and elongation at 72° C. Representative RT-PCR products for each gene were verified by DNA sequencing (data not shown).

Analysis of Differentiation Potency

a) Embryoid Body (EB) Formation

In order to study embryoid body (EB) formation, undifferentiated ES cells were removed from the MEP, dissociated using 0.25% trypsin in PBS with 20% KSR and 2 mM CaCl₂, and cultured in bacterial Petri dishes for 10-21 days using DMEM that was supplemented w with 10% FBS. The medium was changed every two days.

b) In vivo Differentiation Analysis: Teratoma Formation

To examine teratoma formation in mice, between 1-5×10⁶ CMES cells were injected subcutaneously into the abdomen of 5-week-old immunodeficient NOD-scid, γcKO (NOG) mice (Ito et al., Blood 100:3175-3182 (2002). Four to eight weeks after the injection, tumors were resected from the mice. The resected tumors were fixed in buffered formaldehyde, embedded in paraffin blocks, and subjected to immunohistochemical and histological examinations.

c) In vitro Differentiation

i) Neural Cells

Stromal PA6 cells were plated on 12-mm coverslips and grown to semi-confluence. On Day 0, 5×10⁴ ES cells were co-cultured with the PA6 cells on coverslips in Glasgow-MEM (FMEM) medium that was supplemented with 10% KSR, 0.1 mM 2-ME, and 10⁻⁷ M ascorbate. For the first 10 days of co-culture, the medium was changed every two days. On Day 12, the medium was exchanged for GMEM plus N-2 supplement (Invitrogen) that contained 0.1 mM 2-ME and 10⁻⁷ M ascorbate, and the culture was continued until Day 20. The cells were then fixed with 4% paraformaldehyde in PBS.

ii) Hematopoietic Cells

CMES cells were differentiated into hematopoietic cells by EB formation in Iscove's Modified Dulbecco's medium (Invitrogen) that contained 15% FBS, 100 μg/ml transferrin, 10 μg/ml insulin, 50 μg/ml ascorbic acid, and 0.45 mM monothioglycerol. The CMES cells (10⁴ cells per 9-cm dish) were cultured without cytokines for 14-18 days, and then subjected to hematopoietic colony assays. Hematopoietic colonies were examined by growing differentiated ES-derived cells (10⁵ cells) in Methocult GF+medium (StemCell Technologies, Vancouver, Canada), according to the manufacturer's instructions. After 10-14 days, the colony-forming units (CFUs) were counted, and cellular morphology was confirmed microscopically using May-Giemsa Staining of cytospun samples.

Results

Establishment of Common Marmoset ES Cell Lines

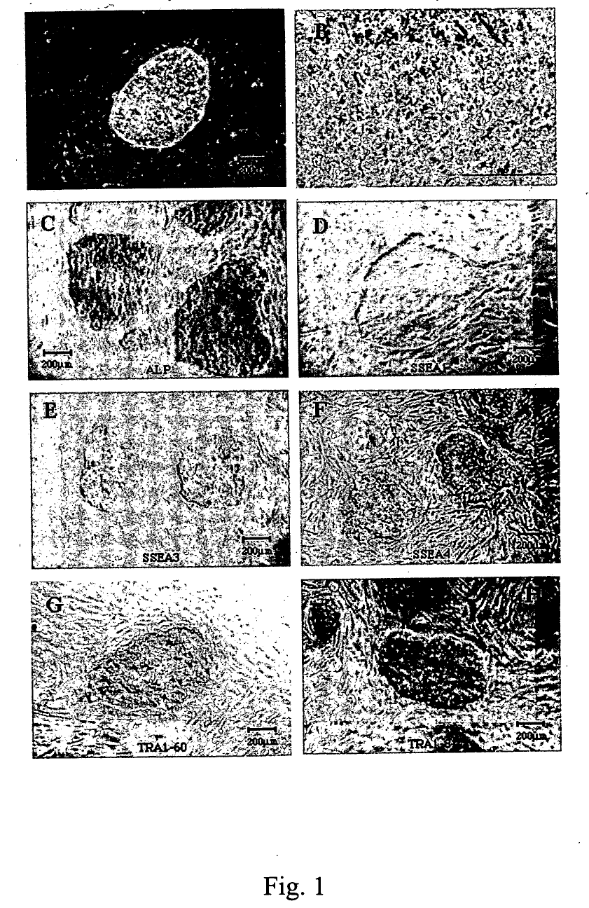

The marmosets ovulated 10.7±1.3 days (n=70) after PGF2α administration. The ovarian cycle control system used made it possible to obtain fertilized eggs every three weeks from the same animals. Sixty immunosurgically isolated ICMs from 70 blastocysts (the ICM isolation rate was 85.7%) were plated on the irradiated MEF layer, and 11 ICMs were cultured for more than ten passages (18.3% derivation rate). To date, 3/11 ICM-derived cells have been cultured for more than one y ear. All ICM-derived cells showed flat, packed, and tight colony morphology and a high nucleus:cytoplasm ratio (FIG. 1A, B). The morphology of these derived cells was very similar to that of reported primate ES cells, including those from humans, and from rhesus and cynomolgus monkeys (Thomson et al., Proc Natl Acad Sci USA 92:7844-7848 (1995); Reubinoff et al., Nat Biotechnol 18: 399-404 (2000); Suemori et al., Dev Dyn 222:273-279 (2001); Thomson et al., Science 282:1145-1147 (1998): Mitalipova et al., Stem Cells 21:521-526 (2003)). It has been reported that primate ES cells exhibit spontaneous differentiation during culture, and that leukemia inhibitory factor does not maintain the ES cells in the undifferentiated state (Reubinoff et al., supra and Suemori et al., supra). The common marmoset ICM-derived cell lines also showed low frequency of differentiation. However, most ICM-derived cells were maintained undifferentiated morphology of the cells. It is known that the quality of FBS is critical to the maintenance of undifferentiated primate ES cells; different lots of FBS from the same manufacturer can vary in this respect. Therefore, the chemically defined serum-free supplement KSR was used for the establishment and culture of the marmoset ES cells. The CMES colonies appeared more packed and tight when KSR was used in the culture; they were flatter in appearance when FBS was used in the medium. To maintain stem cells, the dissociation procedure for cynomolgus ES cells was adopted for subcultue of the marmoset ICM-derived cells (Suemori et al., supra). With this culture system, continuous cultures of CMES cells have been sustained for more than one year.

Characterization of Undifferentiated CMES Cell Lines

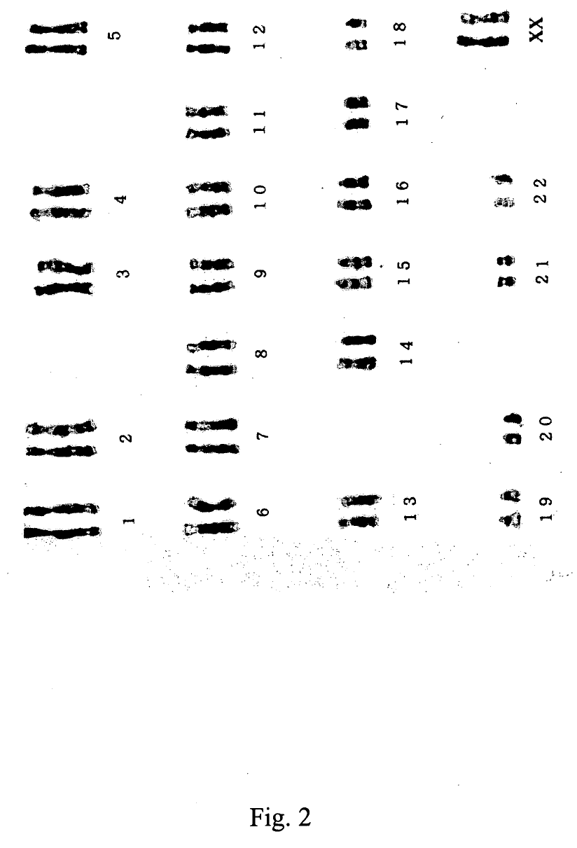

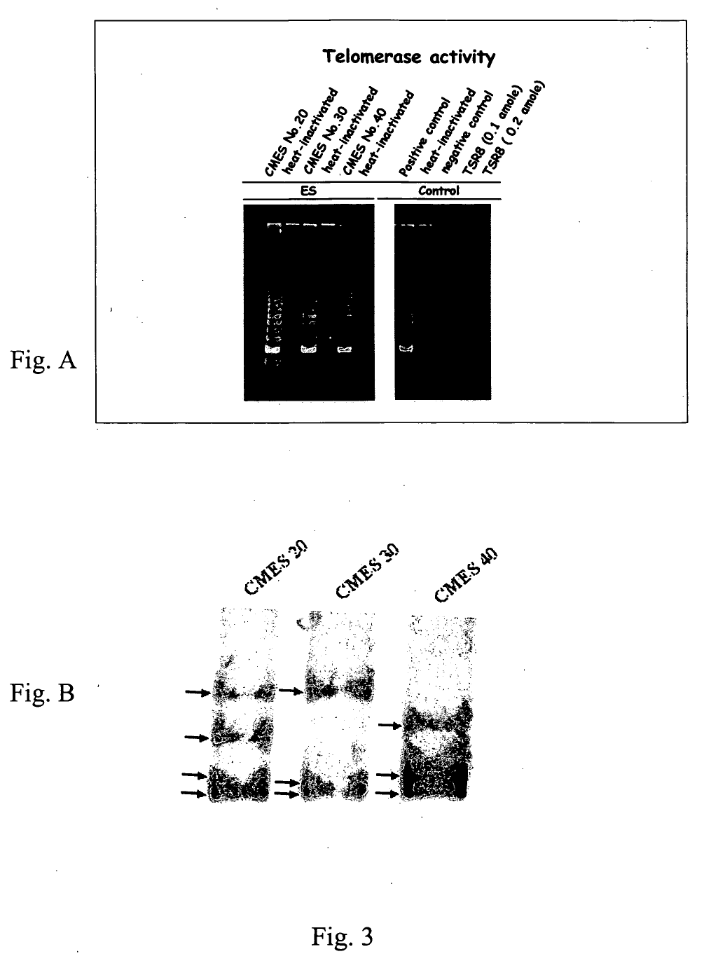

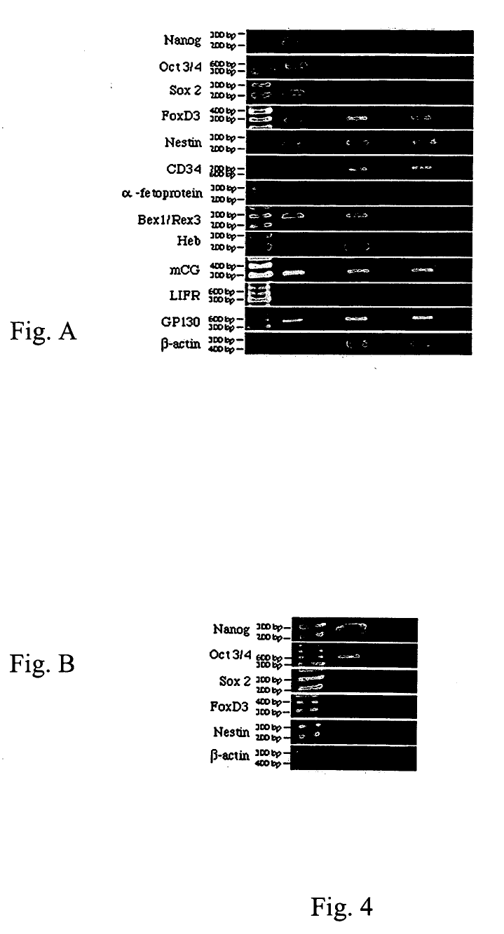

To confirm the undifferentiated status of the ICM-derived cells, all three lines (CMES 20, 30, and 40) were examined for the expression of cell surface markers that are specific for the undifferentiated ES cells. As shown in FIGS. 1C-1G, the ICM-derived cell lines showed alkaline phosphatase activity (FIG. 1C), and expressed SSEA-3 (FIG. 1E), SSEA-4 (FIG. 1F), TRA-1-60 (FIG. 1G), and TRA-1-81 (FIG. 1H), but not SSEA-1 (FIG. 1D). All three cell lines retained the normal 46, XX karyotype (FIG. 2) and Telomerase activity (FIG. 3A). These three ISM-derived cell lines also expressed Nanog, Oct3/4, Sox2, gp130, mCG, HEB, and Bex1/Rex3 mRNA, and low levels of FoxD3 and Nestin mRNA (FIG. 4A). Conversely, LIFR mRNA was not detected by RT-PCR. All three lines showed identical expression patterns of the genes, an indication that these cells are CMES cells. To compare the gene expression patters of fresh ICMs and the ICM-derived cell lines, RT-PCR analysis was conducted using fresh ICMs. As a result, the expression of Nanog, Oct3/4, and Sox2 mRNA was observed, while FoxD3 and Netin mRNA were not detected in fresh ICMs (FIG. 4B).

All three CMES cell lines were examined for MHC-DRB 1 genotype using PCR-based single strand conformation polymorphism (SSCP) methods. As shown in FIG. 3B, the three CMES lines were confirmed, based on different SSCP patterns, as having been established independently. Of these three CMES cell lines, No. 30 and No. 40 were derived from the same parents.

Differentiation Potency

Similarly to other primate ES cells, CMES cells differentiate spontaneously during culturing on MEF. However, the complete differentiation of CMES cells was suppressed by some growth factors of inhibitory factors from MEF. To estimate the degree of differentiation, 50 CMES cell clusters were seeded onto MEF. As a result, 22-74% (n=6) of the colonies (average 42.6%) were morphologically undifferentiated ES cells (data not shown). However, the differentiation rate of each cell was unclear because CMES cells need to be cell clusters to maintain undifferentiated status.

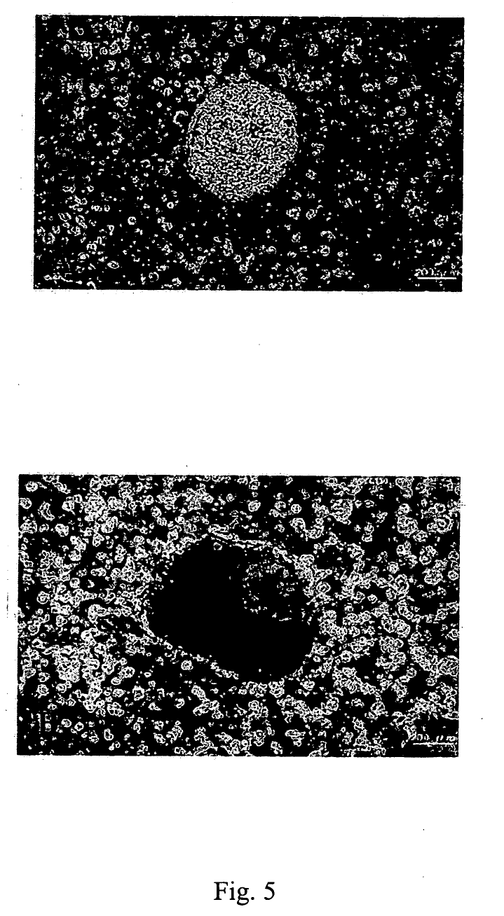

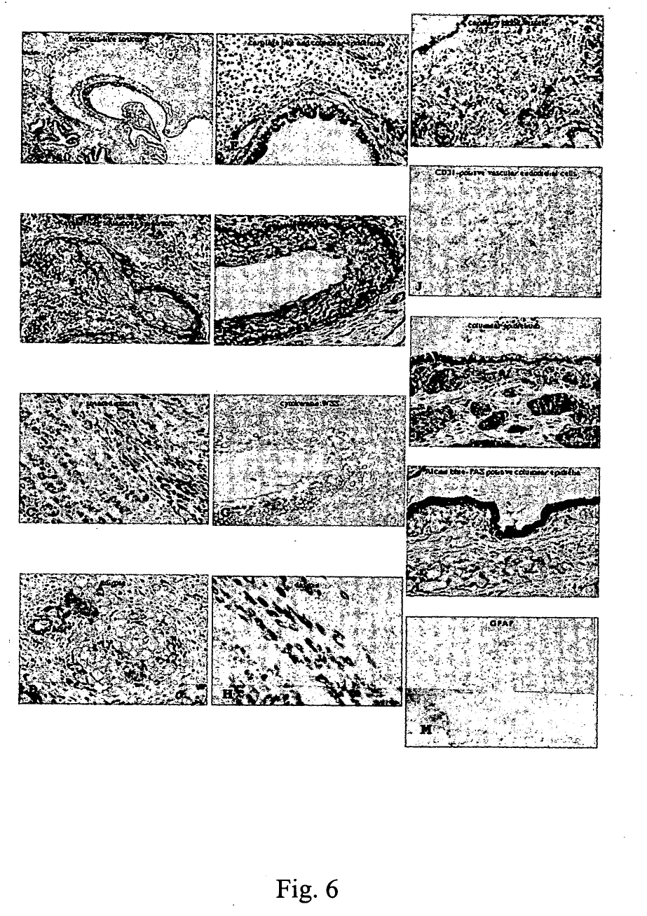

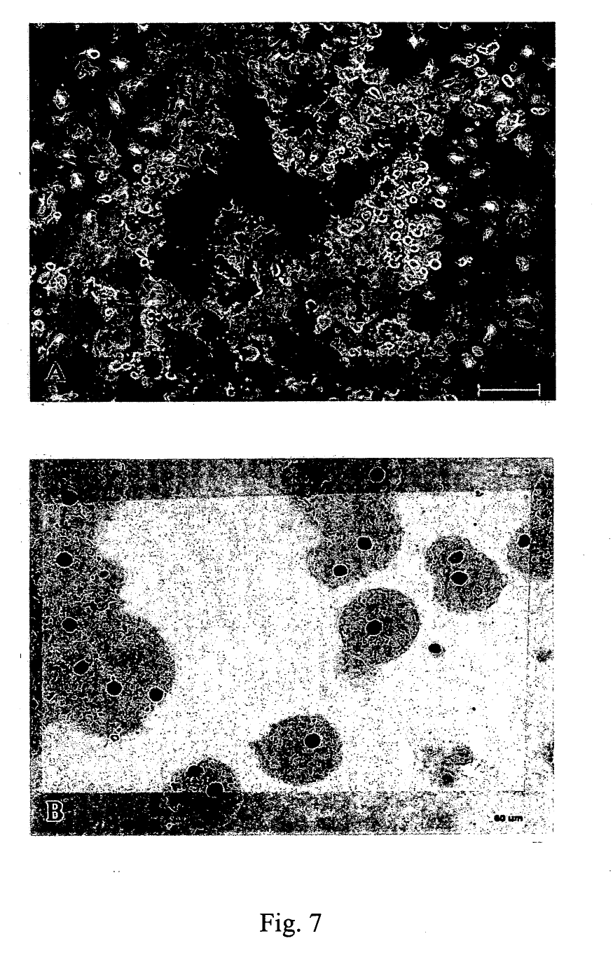

To assess the spontaneous differentiation potency of CMES cells, the formation of EBs and teratomas was examined. The suspension cultures of all three CMES lines formed EBs (FIGS. 5A and B). Simple EBs formed several days after the start of the suspension culture, and cystic EBs formed within two weeks. These EBs expressed mRNA for the Nestin, CD34, and α-fetoprotein genes, which are marker genes for the three germ layers and mCG, Bex1/Rex3 and Heb, which are marker genes for trophectoderm (FIG. 4A). Furthermore, expression of LIFR and gp130 was observed. However, Nenog, Oct 3/4, and Dox 2 gene expression was shut off after two weeks of EB culture. In contrast, the expression level of FoxD3 and EBs was greater than in undifferentiated CMES. To examine the differentiation potency in more detail, cells of CMES 20 were injected subcutaneously into five immunodeficient NOG mice (Ito et al., supra). Eight weeks after injection, subcutaneous tumors were rescued from these mice and subjected to histological analysis. The tumor formation rate was 100% (5/5). The tumors were found to be teratomas that consisted of embryonic germ layers of ectodermal, mesodermal, and endodermal tissues (FIG. 6A-M). Teratomas formed in all five NOG mice (100% teratoma formation rate). In the teratomas, the ectodermal tissue consisted of keratinized epidermis (FIGS. 6B, 7F, and 6G) and neuronal cells (FIG. 6M), the mesodermal tissue was comprised of muscle (FIGS. 6C and 6H) and blood vessels (FIGS. 6I and 6J), and the endodermal tissue contained columnar epithelium (FIGS. 6A, 6K, and 6L). Furthermore, cartilage-like tissue (FIGS. 6A, 6D, and 6E) and adipose-like tissue (FIG. 6F) were also observed. These blood vessels were distinguished from murine blood vessels by immunohistochemical staining with human anti-CD31 antibody. Bronchus-like structures and gut-like structures were occasionally found in the teratomas (FIGS. 6A, 6K, and 6L). Differentiation was confirmed by immunohistochemical analysis with several tissue-specific antibodies. As evidence for ES cell differentiation into ectodermal cells, the glial fibrillary acidic protein (GFAP)-positive cells were observed as neuronal cells (FIG. 6M) and the keratinized epidermis-like structures in the teratomas expressed wider specific (WSS) keratin (FIG. 6G). The teratomas differentiated frequently into mesodermal tissues, such as muscle, blood vessels, and cartilage. The muscle-like structure showed desmin expression, and CD3 1-positive cells were located in the hemangioendothelium of the blood vessel-like structures (FIG. 6J). The presence of the gut-like structures suggests endodermal differentiation. Alcian blue and PAS staining revealed mucus secretion from the columnar epithelium (FIG. 6L).

To investigate the in vitro differentiation potency of the CMES cells, the measurement of stromal cell-derived inducing activity (SDIA) was performed. After culture on PA6 cells for 20 days, extensive neuritis appeared in the majority of the primate ES cell colonies (67%, n=30), which contained a large number of post-mitotix neurons that were positive for class III β-tubulin (not shown). SDIA has been reported to induce the production of tyrosine hydroxylase (TH)-positive dopaminergic neurons in mouse, cynomolgus monkey, and human ES cells (Kawasaki et al., Neuron 28:31-40 (2000); Kawasaki et al., Proc Natl Acad Sci USA 99:1580-1585 (2002); and Perrier et al., Proc Natl Acad Sci USA 101:12543-12548 (2004)). Therefore, the marmoset cells were tested for similar activities. After two weeks of induction, 14% of the class III β-tubulin positive post-mitotix neurons were TH-positive at the cellular level (N=50). These cells were plated from trypsinized ES cells at passage. Most of the cells were derived from single cells, but only 15% of them expressed the TH protein. In the teratoma-like tumor induced by subcutaneous transplantation into NOG mice, some βIII tubulin-positive cells were found in colonies. Among βIII positive neurons, TH-positive neurons were found in 21% (n=1 13; data not shown).

To induce hematopoietic cells, EB formation was allowed to proceed in the cytokine-free medium, and CFU assays were performed. CFU-M_monocyte/macrophage) colonies were mainly observed under these conditions (FIG. 7A), and the main population of macrophages was confirmed microscopically using May-Giemsa staining of cytospun preparations (FIG. 7B).

### DISCUSSION

In this study, an embryo collection system has been developed, that ensures a stable supply of common marmoset embryos for future production of transgenic or gene knockout marmosets. Our results show that marmoset ovulation is not disturbed, even after continuous administration of the PGF2α analog, cloprostenol. Thus, embryo collection from the same animal was carried out routinely, every three weeks, using cloprostenol administration. Relying on this system, CMES cell lines were established. In this study, the reported methodology of immunosurgery was used (Solter and Knowles, Proc Natl Acad Sci USA 72:5099-5102 (1975)). Although there was a high ICM isolation rate (85.7%) from blastocysts, the CMES derivation rate was 18.3%, which is comparable to previous reports on other primate ES cell lines, including 35.7% for human and 12.5% for cynomolgus monkey. The derivation rate of CMES lines was considered to be dependent on the expansion procedure used for the cultured ICMs, including the in vitro developmental ability of embryos or the passage technique used for expanded ICMs.

The results of immunohistochemical analysis, enzymatic activity assays, RT-PCR analysis, and karyotype analysis show that the CMES cell lines maintain their undifferentiated status for an extended period of time. The RT-PCR results indicate that the gene expression patterns of undifferentiated CMES cells are similar to that of human ES cells and different from those of mouse ES cells. In undifferentiated CMES cells, the expression patterns of Ect3/4, Nanog, Sox2, mCG, HEB, and Bex1/Rex3 gene mRNA were identical to human or marmoset ES cells (Thomson et al., Biol Reprod 55:254-259 (1996); Ginis et al., Dev Biol 269:360-380 (2004)). In contrast, a very low level of FoxD3 expression was observed in undifferentiated CMES. In fresh ICMs, the expression of Nanog, Oct3/4, and Sox2 was observed, while that of FoxD3 and Nestin was not. The presence of Nanog, Oct3/4, and Sox2 mRNA expression in both fresh ICMs and CMES cells suggests that these genes are required to maintain stemness of cells in vitro and in vivo. Since FoxD3 and Nestin were expressed at very low levels in undifferentiated CMES cells, it is possible that these genes in fresh ICMs are expressed under the detection level of the present RT-PCR analysis. Another possibility is that the FoxD3 and Nestin genes were amplified from spontaneously differentiated CMES cells in the cultured CMES. The latter possibility is supported by increased FoxD3 gene expression in EBs. As the FoxD3 gene is expressed in murine undifferentiated ES cells, the different FoxD3 expression patterns in primate and murine ES cells suggest that FoxD3 plays a different role in respective ES cells. The expression of mCG and Bex1/REX3 reflects the ability of CMES to differentiate into trophectodermal cells.

The molecular mechanisms that maintain undifferentiated primate ES cells are largely unknown, and MEF is essential to maintain undifferentiated primate cells. CMES cells also differentiated spontaneously on MEF at low frequency. The expression of gp130 and the absence of LIFR in CMES cells were identical to human ES cells. These results indicate that maintaining undifferentiated CMES cells is not dependent on LIF signals. Gowever, the expression of gp130 suggested that other gp130-STAT3 signals, such as interleukin (IL)-6, oncostatin M, or L-11, play a role in CMES cell proliferation and differentiation. The MEF dependency of primate ES cells is considered one significant obstacle to using human ES cells for stem therapy. Elucidation of the molecular mechanisms for the maintenance or development of MEF-free culture systems of undifferentiated primate ES cells is one of the important themes of ES cell research. Combined, these results suggest that the cellular characteristics and activities of CMES cells are similar to those of human and other primate ES cells.

The spontaneous differentiation abilities of the CMES cells were verified by EB formation (FIG. 5) and teratoma formation (FIG. 6). RT-PCR analysis of EBs showed that the CMES cells differentiated into three germ layers in vitro. Furthermore, when the CMES cells were transplanted subcutaneously into immunodeficient NOG mice, teratomas that consisted of three-dimensional tissue structures were formed with high efficiency (100%). When SCID mice were used for the teratoma formation experiment, the teratoma formation rate was also 100% (two of two NOD/SCID mice, data not shown). Therefore, the formation of teratomas indicates that CNES cells have multipotent differentiation ability. The teratomas were also examined by immunohistochemistry using several tissue-specific antibodies. The most frequently observed tissue type in the teratomas was mesodermal, which included cartilage (FIGS. 6A, D, E), muscle (FIGS. 6C and 6H), and blood vessels (FIG. 6I). Although the frequency of endodermal tissue differentiation was lower than the frequencies of extodermal and mesodermal tissue differentiation, endodermal tissue differentiation was clearly demonstrated with at least one of the CMES cell lines (No. 20). Interestingly, Alcian blue and PAS staining of columnar epithelium showed the secretion of mucus from the cells, which indicates that CMES cells can differentiated into functional endodermal cells (FIG. 6L). Teratoma formation was not demonstrated for the marmoset ES cell line cj 11) established by Thomson et al. (Biol Reprod 55:254-259 (1996)). Both CMES and cj 11 showed similar morphology and identical marker expression patterns. However, when the teratoma formation was examined with a marmoset ES cell line produced from the WiCell Research Institute (Madison, Wis.), fibrosarcomas, but no teratomas, were obtained in NOD/SCID mice (data not shown).

SDIA caused CMES cell lines No. 20 and 40 to differentiate into TH-positive neurons in vitro (data not shown). In vitro differentiation into hematopoietic cells from EBs was measured in colony assays. Although other types of hematopoietic colonies were not seen in all experiments (n>7), other conditions, such as gene-transfer method, make it possible to induce various hematopoietic colonies from CMES cells, which suggests that CMES cells have the ability to differentiate into multiple hematopoietic lineages. These results support the finding that CMES cells have the capacity to differentiate into functional cells both in vitro and in vivo. Therefore, CMES cells can be used in pre-clinical studies aimed at developing regenerative therapies or medicines.

The teratoma formation and in vitro differentiation experiments show the strong differentiating potency of the present CMES cell lines; in addition, this is the first report to demonstrate the pluripotency of CMES cells. Indeed, in vitro formation of embryoid body and teratoma suggest totipotency of the present CMES cell lines. The present CMES cell lines find utility in establishing a pre-clinical animal model system for predicting the safety and efficacy of regenerative therapies using human ES cells. Chimerism and the germ line transmission ability of CMES require further studies. If CMES cells do not transmit into the germ line, somatic cell nuclear transfer from the chimera or gametogenesis from CMES is a way to address this issue (Toyooka et al., Proc Natl Acad Sci USA 100:11457-11462 (2003); Hubner et al., Science 300:1251-1256 (2003); Simerly et al., Dev Biol 276:237-252 (2004)).

Recently, various types of cells that have been differentiated from human ES cells or non-human ES cells have been reported (see, e.g. Kawasaki et al., Proc Natl Acad Sci USA 99:1580-1585 (2002); Assady et al., Diabetes 50:1691-1697 (2001); Schuldiner et al., Brain Res 913:201-205 (2001); Umeda et al., Development 131:1869-1870 (2004); Haruta et al., Invest Ophthalmol Vis Sci 45:1020-1025 (2004); Mizuseki et al., Proc Natl Acad Sci USA 100:5828-5833 (2003); Kuo et al., Biol Reprod 68:1727-1735 (2003)). For example, hematopoietic cells, dopaminergic neurons, and insulin-producing cells have been generated from human or primate ES cells. However, reports of transplantation of these differentiates ES cells into non-human primates are very rare (Nara et al., Nippon Rinsho 62:1643-1647 (2004)). There are various difficulties involved in using non-human primates as experimental animals. Fr example, rhesus or cynomolgus monkeys in which the ES cell line has already been established are too expensive and cumbersome. Furthermore, immunological incompatibility between ES cells and animals may represent a significant obstacle to ES cell transplantation. Taking this situation into consideration, the common marmoset has significant advantages, such as low cost and ease of maintenance. Importantly, marmosets are immunogenetically closed because they have been bred in large closed colonies. Thus, the common marmoset and CMES cells provide an excellent experimental model system for studies into the mechanism of cell differentiation, as well as for the development of regenerative therapies, using human ES cells.

All references cited throughout this disclosure are hereby expressly incorporated by reference.

What is claimed is:

1. A purified preparation of embryonic stem cells derived from common marmoset (Callithrix jacchus), which (i) is capable of prolonged undifferentiated proliferation in vitro, (ii) maintains, during prolonged culture, a karyotype in which all the chromosomal characteristics of common marmoset are present without noticeable alteration; (iii) is capable of differentiation into all three embryonic germ layers (ectoderm, endoderm and mesoderm) even after prolonged culture; and (iv) is capable of teratoma formation in vivo.

2. The purified preparation of claim 1 wherein said embryonic stem cells are totipotent.

3. The purified preparation of claim 1 wherein the stem cells are positive for the SSEA-3, SSEA-4, TRA1-60, and TRA1-81 markers, and negative for the SSEA-1 marker.

4. The purified preparation of claim 3 wherein the stem cells additionally express one or more of the cell markers selected from the group consisting of Nanog, Oct3/4, Sox2, gp130, mCG, HEB and Bex1/Rex3.

5. The purified preparation of claim 4 wherein the stem cells exhibit high telomerase activity.

6. The purified preparation of claim 1 wherein the stem cells are capable of undifferentiated proliferation in vitro for at least 3 months.

7. The purified preparation of claim 1 wherein the stem cells are capable of undifferentiated proliferation in vitro for at least 6 months.

8. The purified preparation of claim 1 wherein the stem cells are capable of undifferentiated proliferation in vitro for at least one year.

9. The purified preparation of claim 1 capable of differentiation into all three embryonic germ layers after continuous culture for at least 3 months.

10. The purified preparation of claim 1 capable of differentiation into all three embryonic germ layers after continuous culture for at least 6 months.

11. The purified preparation of claim 1 capable of differentiation into all three embryonic germ layers after continuous culture for at least one year.

12. A cell line developed from embryonic stem cells of common marmoset (Callithrix jacchus), which (i) is capable of prolonged undifferentiated proliferation in vitro, (ii) maintains, during prolonged culture, a karyotype in which all the chromosomal characteristics of common marmoset are present without noticeable alteration; and (iii) is capable of differentiation into all three embryonic germ layers (ectoderm, endoderm and mesoderm) even after prolonged culture, and (iv) is capable of teratoma formation in vivo.

13. The cell line of claim 12 which is positive for the SSEA-3, SSEA-4, TRA1-60, and TRA1-81 markers, and negative for the SSEA-1 marker.

14. The cell line of claim 13 which additionally expresses one or more of the cell markers selected from the group consisting of Nanog, Oct3/4, Sox2, gp130, mCG, HEB and Bex1/Rex3.

15. A method of making a genetically modified common marmoset (Callithrix jacchus), comprising
(a) introducing a mutation into a stem cell according to claim 1, and
(b) transplanting said stem cell into said common marmoset.

16. The method of claim 15 further comprising the step of analyzing the differentiation and proliferation of said stem cells in said common marmoset.

17. The assay method of claim 16 wherein said stem cells have a foreign gene introduced thereinto.

18. A common marmoset (Callithrix jacchus) carrying a genetic modification resulting from the introduction into said common marmoset genetically modified common marmoset embryonic stem cells which (i) are capable of prolonged undifferentiated proliferation in vitro, (ii) maintain, during prolonged culture, a karyotype in which all the chromosomal characteristics of common marmoset are present without noticeable alteration; (iii) are capable of differentiation into all three embryonic germ layers (ectoderm, endoderm and mesoderm) even after prolonged culture; and (iv) are capable of teratoma formation in vivo.

19. The common marmoset of claim 18 wherein said stem cells are totipotent.

20. An immunodeficient rodent having embryonic stem cells of common marmoset (Callithrix jacchus) transplanted therein, wherein said cells (i) are capable of prolonged undifferentiated proliferation in vitro, (ii) maintain, during prolonged culture, a karyotype in which all the chromosomal characteristics of common marmoset are present without noticeable alteration; (iii) are capable of differentiation into all three embryonic germ layers (ectoderm, endoderm and mesoderm) even after prolonged culture, and (iv) are capable of teratoma formation in vivo.

21. The immunodeficient rodent of claim 20 wherein said stem cells are totipotent.

22. The method of claim 20 wherein said rodent is a mouse or a rat.

23. The method of claim 22 wherein said mouse is selected from the group consisting of athymic nude mouse, C.B-17/severe combined immunodeficiency (scid) mouse and NOD/SCID mouse.

24. The method of claim 23 wherein said mouse is a NOD/SCID/γ_{c}^{null} mouse.

25. The mouse of claim 23 wherein said mouse additionally carries a transgene.

26. The mouse of claim 25 wherein said mouse is an animal model of a disease or condition susceptible to cell based therapy.

27. The method of claim 26 wherein said disease or condition is selected from the group consisting of Parkinson's disease, diabetes, traumatic spinal cord injury, Purkinje cell degeneration, Duchenne's muscular dystrophy, heart disease, vision loss and hearing loss.

28. The method of claim 20 wherein said embryonic stem cells are in the form of embryoid bodies.

29. The method of claim 20 wherein said embryonic stem cells are in a differentiated form.

30. The method of claim 29 wherein the differentiated stem cells are selected from the group consisting of muscle cells, nerve cells, and blood cells.


SEQUENCE LISTING (28 sequences)

SEQ ID NO: 1 - DNA, 20 bp, Artificial Sequence
  Synthetic Oligonucleotide Primer
ctctccgcggcactaggaac

SEQ ID NO: 2 - DNA, 21 bp, Artificial Sequence
  Synthetic Oligonucleotide Primer
gcacgtttcttggagtatagc

SEQ ID NO: 3 - DNA, 21 bp, Artificial Sequence
  Synthetic Oligonucleotide Forward Primer
aaacagaagaccagaactgtg

SEQ ID NO: 4 - DNA, 21 bp, Artificial Sequence
  Synthetic Oligonucleotide Reverse Primer
agttgtttttctgccacctct

SEQ ID NO: 5 - DNA, 20 bp, Artificial Sequence
  Synthetic Oligonucleotide Forward Primer
cctgggggttctatttggga

SEQ ID NO: 6 - DNA, 20 bp, Artificial Sequence
  Synthetic Oligonucleotide Reverse Primer
tttgaatgcatgggagagcc

SEQ ID NO: 7 - DNA, 21 bp, Artificial Sequence
  Synthetic Oligonucleotide Forward Primer
cgacgacgggctggaggagaa

SEQ ID NO: 8 - DNA, 20 bp, Artificial Sequence
  Synthetic Oligonucleotide Reverse Primer
atgagcgcgatgtacgagta

SEQ ID NO: 9 - DNA, 20 bp, Artificial Sequence
  Synthetic Oligonucleotide Forward Primer
agaaccccaagatgcacaac

SEQ ID NO: 10 - DNA, 20 bp, Artificial Sequence
  Synthetic Oligonucleotide Reverse Primer
gggcagcgtgtacttatcct

SEQ ID NO: 11 - DNA, 20 bp, Artificial Sequence
  Synthetic Oligonucleotide Forward Primer
agcctgtcacctggaaatgc

SEQ ID NO: 12 - DNA, 21 bp, Artificial Sequence
  Synthetic Oligonucleotide Reverse Primer
cgtgttgtcttgctgaatggc

SEQ ID NO: 13 - DNA, 20 bp, Artificial Sequence
  Synthetic Oligonucleotide Forward Primer
gccctgaccactccagttta

SEQ ID NO: 14 - DNA, 20 bp, Artificial Sequence
  Synthetic Oligonucleotide Reverse Primer
ggagtcctggatttccttcc

SEQ ID NO: 15 - DNA, 25 bp, Artificial Sequence
  Synthetic Oligonucleotide Forward Primer
gctggattgtctgcaggatggggaa

SEQ ID NO: 16 - DNA, 25 bp, Artificial Sequence
  Synthetic Oligonucleotide Reverse Primer
tcccctgaagaaaattggttaaaat

SEQ ID NO: 17 - DNA, 19 bp, Artificial Sequence
  Synthetic Oligonucleotide Forward Primer
ccctgtgtgtgtcgccttt

SEQ ID NO: 18 - DNA, 20 bp, Artificial Sequence
  Synthetic Oligonucleotide Reverse Primer
ctaatggagggtctgctggc

SEQ ID NO: 19 - DNA, 20 bp, Artificial Sequence
  Synthetic Oligonucleotide Forward Primer
acaggcaaggatgagagaag

SEQ ID NO: 20 - DNA, 20 bp, Artificial Sequence
  Synthetic Oligonucleotide Reverse Primer
cccacgtaaacaagtgacag

SEQ ID NO: 21 - DNA, 26 bp, Artificial Sequence
  Synthetic Oligonucleotide Forward Primer
actgaaacaaagaaaggatgaaaacc

SEQ ID NO: 22 - DNA, 26 bp, Artificial Sequence
  Synthetic Oligonucleotide Reverse Primer
ccctttctatcttctgttcagggttc

SEQ ID NO: 23 - DNA, 20 bp, Artificial Sequence
  Synthetic Oligonucleotide Forward Primer
aaacagaacagcatccagtc

SEQ ID NO: 24 - DNA, 20 bp, Artificial Sequence
  Synthetic Oligonucleotide Reverse Primer
agttgaggcatctttggtcc

SEQ ID NO: 25 - DNA, 19 bp, Artificial Sequence
  Synthetic Oligonucleotide Forward Primer
tttcttggcatttaccagg

SEQ ID NO: 26 - DNA, 19 bp, Artificial Sequence
  Synthetic Oligonucleotide Reverse Primer
gctattttggaaggtggtg

SEQ ID NO: 27 - DNA, 20 bp, Artificial Sequence
  Synthetic Oligonucleotide Forward Primer
tcctgaccctsaagtacccc

SEQ ID NO: 28 - DNA, 20 bp, Artificial Sequence
  Synthetic Oligonucleotide Reverse Primer
gtggtggtgaagctgtagcc